(19) United States
(12) Patent Application Publication
(10) Pub. No.: US 2009/0029393 A1
(43) Pub. Date: Jan. 29, 2009

(54) METHOD FOR PREPARING ANTIBODIES SELECTIVE FOR ACTIVATING FC RECEPTORS

(75) Inventors: Jean-Luc Teillaud, Paris (FR); Sylvie Jorieux, Villeneuve D'Ascq (FR); Sophie Siberil, Paris (FR); Renee Menez, Magny Les Hameaux (FR); Enrico Stura, Rambouillet (FR); Frederic Ducancel, Longjumeau (FR)

Correspondence Address: MARSH, FISCHMANN & BREYFOGLE LLP, 8055 East Tufts Avenue, Suite 450, Denver, CO, 80237, US

(73) Assignee: LFB Biotechnologies, LES ULIS (FR)

(21) Appl. No.: 12/097,022

(22) PCT Filed: Dec. 15, 2006
(86) PCT No.: PCT/FR2006/002748

§ 371 Date: Oct. 9, 2008

(30) Foreign Application Priority Data
Dec. 16, 2005 (FR) 0512812

(51) Int. Cl. G01N 33/566; C07K 16/18

(52) U.S. Cl. 435/7.23; 435/7.21; 435/7.2; 435/7.24; 530/389.1

(57) ABSTRACT

The present invention relates to a method for preparing an antibody selective for activating antibody Fc region receptors (FcRs) comprising an ITAM motif or motifs (immunoreceptor tyrosine-based activation motif), comprising the steps of obtaining monoclonal antibodies from a hybridoma, from a heterohybridoma or from any animal, plant or human cell line, replacing each of the His 310 and His 435 residues (Cabat numbering) of the Fc region of said antibody with a residue chosen from lysine, alanine, glycine, valine, leucine, isoleucine, proline, methionine, tryptophan, phenylalanine, serine or threonine, and then selecting the antibodies for which the binding to inhibitory FcRs comprising ITIM motifs (immunoreceptor tyrosine-based inhibition motif) is decreased by at least 30%, preferably by at least 50%, 70%, 80% or else by at least 90% relative to the same antibody having a natural Fc region. The present invention also relates to the use of an antibody derived from the method of the invention in the production of a medicament for use in particular in the treatment of cancer and of infectious pathologies.

The present invention relates to a method for preparing an antibody selective for activating antibody Fc region receptors (FcRs) comprising an ITAM motif or motifs (immunoreceptor tyrosine-based activation motif), comprising the steps of obtaining monoclonal antibodies from a hybridoma, from a heterohybridoma, or from any animal, plant or human cell line, replacing each of the His 310 and His 435 residues (Kabat numbering) of the Fc region of said antibody with a residue chosen from lysine, alanine, glycine, valine, leucine, isoleucine, proline, methionine, tryptophan, phenylalanine, serine or threonine, then selecting the antibodies for which the binding to inhibitory FcRs comprising ITIM motifs (immunoreceptor tyrosine-based inhibition motif) is reduced by at least 30%, preferably by at least 50%, 70%, 80% or also by at least 90% relative to the same antibody possessing a native Fc region. The present invention also relates to the use of an antibody originating from the method of the invention in obtaining a medicament intended in particular for the treatment of cancer and infectious pathologies.

### INTRODUCTION AND PRIOR ART

Numerous antibody preparations for therapeutic use, of plasmatic or biotechnological origin, are currently on the market, or in the clinical development phase. Their properties are exploited in order to obtain therapeutic tools capable of binding specifically to their target, and effectively recruiting immune cells.

These last few years, research has been directed towards improving the effectiveness of antibodies, and more particularly towards the manipulation of their constant Fc region. It is the latter which is responsible for the “effector” properties of the antibodies, as it allows the mobilization of the effector immune cells and complement molecules. This ability is made possible by the presence, on certain immune cells, of glycoproteins, the Fc receptors or FcRs. These receptors are capable of binding to the constant region of the antibodies, in particular once the latter have bound, by their variable region, the target antigen. On contact with these cells, the antibodies trigger different cell mechanisms such as phagocytosis and ADCC (Antibody-Dependent Cell-mediated Cytotoxicity).

However, different human FcR classes exist. The latter are encoded by eight human genes, all located on chromosome 1. Certain of these genes exhibit an allelic polymorphism generating different receptor allotypes then having different IgG binding properties (Hulett M. D. & Hogarth P. M., Advances in Immunology, vol. 57, pp. 1-127, 1994) leading to differential effector properties (Carton et al. (2002) Blood, vol. 99, no. 3, 754-758). The main human FcR classes identified are FcγRI (CD64, possessing the A and B isoforms), the FcγRII receptor (CD32, possessing the A and B isoforms) and the FcγRIII receptor (CD16, possessing the A and B isoforms). The FcyRI and FcγRIII receptors, as well as the FcγRIIA receptor are receptors qualified as “activating”, as their activation, using the ITAM motifs comprised by their sequences or the sequences of associated chains, allows the triggering of effector functions such as the lysis of the target cells. Conversely, the FcγRIIB receptors are qualified as “inhibiting” receptors, as they inhibit the transduction pathways of the activating receptors via their ITIM motifs comprised by their sequences and negatively modulate the effector mechanisms such as those of the ADCC induced via the activating FcRs or other surface molecules such as the B-cell antigen receptor (BCR) or growth factor receptors such as c-kit.

Therefore, the diversity of the FcR receptors, in particular the existence of activating FcRs and inhibitory FcRs expressed on the same cells is capable of modulating the effectiveness of the therapeutic antibodies, the engagement of inhibitory FcRs counter-balancing the engagement of the activating FcRs.

It was therefore the intention of the Applicant to provide a therapeutic antibody the effectiveness of which, i.e. the ability to activate the effector immune cells, is not at all or only slightly modulated by the diversity of the FcR receptors, in particular by the inhibitory FcRs.

The Applicant, in the document WO 2005/040216, describes antibodies the primary sequence of which is modified, being particularly useful in IgG4 replacement therapy, or for preventing graft rejections, or also as anti-tetanus, anti-diphtheria agents or directed against certain pathogenic agents or derived toxins.

However, the Applicant surprisingly found that the antibody thus modified retained its ability to induce an effector function dependent on the activating receptors, whereas it no longer possesses the ability to recruit the inhibitory receptors.

The Applicant therefore sought to develop a method making it possible to provide therapeutic antibodies the effectiveness of which, i.e. the ability to activate the effector immune cells, is not at all or only slightly, modulated by the diversity of the FcR receptors, in particular by the inhibitory FcRs.

### DETAILED DESCRIPTION OF THE INVENTION

The invention relates firstly to a method for preparing an antibody possessing the ability to recruit the activating FcRs, but the ability of which to recruit inhibitory FcRs is reduced relative to the same antibody possessing a native Fc region (i.e. the Fc region of which selectively binds the activating antibody Fc region receptors (FcRs)), comprising the following steps:
a) obtaining monoclonal antibodies from a hybridoma, heterohybridoma, or any animal, plant or human cell line,
b) replacing each of the His 310 and His 435 residues (Kabat numbering) of the Fc region of the antibody with a residue chosen from lysine, alanine, glycine, valine, leucine, isoleucine, proline, methionine, tryptophan, phenylalanine, serine or threonine,
c) selecting the antibodies for which the binding of the Fc region to inhibitory FcRs is reduced by at least 30%, preferably by at least 50%, 70%, 80% or also by at least 90% relative to the same antibody possessing a native Fc region.

For the purposes of the invention, by “antibody” is meant any antibody, whatever its specificity and its isotype, provided that it comprises an Fc region or a region possessing the same functions and the same characteristics as the Fc region. Thus, it can be a whole antibody or an antibody fragment, for example an Fc antibody fragment, or a fusion molecule comprising an Fc region at one of its ends. Moreover, the antibodies utilized in the method according to the invention can be IgGs, i.e. any IgG1, any IgG2, any IgG3 (G3m(s) or G3m(st) allotypes) and any IgG4, IgM, IgE, IgA or IgD, or also a mixture of one or more of these. Moreover, the antibodies utilized in the method according to the invention can be monoclonal or polyclonal. In the case where they are monoclonal antibodies, these antibodies can be chimeric, humanized, human or of animal origin.

Moreover, the word “antibody” also denotes an antibody composition, composed of molecules of antibodies possessing the same amino acid sequence, expressed in the same biological system, and comprising at least one excipient or pharmaceutically acceptable vehicle such that the composition can be formulated in order to allow pharmaceutical administration, for a prophylactic and/or therapeutic purpose.

At the end of step b), the sequences coding for the modified antibody of the invention are expressed in a suitable biological system (stage b′).

For the purposes of the invention, by “same antibody” is meant a non-modified antibody according to Step b) of the method of the invention, and produced in the same biological production system as the modified antibody of the invention. The “same antibody” possesses the same native primary sequence (apart from the His 310 and His 435 residues, which have been modified in the antibody of the invention) and has been subjected to the same post-translational modifications as the antibody obtained by the method of the invention, since it has been produced in the same biological system.

For the purposes of the invention, the antibodies of Step a) are obtained in the form of monoclonal antibody compositions. Each monoclonal antibody composition is composed of molecules of antibodies possessing the same amino acid sequence, and therefore the same specificity, expressed in the same biological system. These compositions can, if appropriate, contain at least one excipient or pharmaceutically acceptable vehicle such that these compositions can be formulated in order to allow pharmaceutical administration, for a prophylactic and/or therapeutic purpose.

The antibodies of Step a) comprise a native Fc region, i.e. they possess histidine residues at positions 310 and 435 (Kabat numbering) of their Fc region.

Preferably, the Fc region of such antibodies binds both to the activating receptors and to the inhibitory receptors. For the purposes of the invention, by “native Fc region of the antibody” is meant any Fc region not modified by any chemical or biotechnological methods on the His 310 and His 435 residues. More particularly, by “native Fc region of the antibody” is meant any Fc region the His 310 and His 435 residues of which have not been replaced, by chemical or biotechnological methods, with a residue chosen from lysine, alanine, glycine, valine, leucine, isoleucine, proline, methionine, tryptophan, phenylalanine, serine or threonine.

By way of example, the antibody utilized in the invention can be chosen from anti-Ep-CAM, anti-KIR3DL2, anti-EGFR, anti-VEGFR, anti-HER1, anti-HER2, anti-GD, anti-GD2, anti-GD3, anti-CD20, anti-CD-23, anti-CD-25, anti-CD30, anti-CD33, anti-CD38, anti-CD44, anti-CD52, anti-CA125 and anti-Protein C, anti-HLA-DR, the anti-virals: HBV, HCV, HIV and RSV, and more particularly from the antibodies of Table 1 hereafter:

TABLE 1

| Name and | | | |
|---|---|---|---|
| trademark of | | | |
| the antibody | Company | Target | Indication |
| Edrecolomab | Centocor | anti-EpCAM | Colorectal cancer |
| PANOREX | | | |
| Rituximab | Idec | anti-CD20 | Cell lymphoma |
| RITUXAN | Licensed to Idec Genentech/ | | |
| | Hoffman la Roche | | thrombocytopenic purpura |
| Trastuzumab | Genentech | anti-HER2 | Mammary carcinoma |
| HERCEPTIN | Licensed to Hoffman la | | ovarian cancer |
| | Roche/Medimmune | | |
| Palivizumab | Licensed to Abbott | | RSV |
| SYNAGIS | | | |
| Alemtuzumab | BTG | anti-CD52 | Leukaemia |
| CAMPATH | Licensed to Schering | | (B-CLL) |
| ibritumomab | IDEC | anti-CD20 | NHL |
| tiuxetan | Licensed to Schering | | |
| ZEVALIN | | | |
| Cetuximab | Merck/BMS/ | anti-EGFR | Ovarian, colorectal, |
| IMC-C225 | Imclone | | mammary cancers |
| Bevacizumab | Genentech/ | anti-VEGFR | Colorectal cancers |
| AVASTIN | Hoffman la Roche | | |
| Epratuzumab | Immumedics/ | anti-CD22 | Cancers: |
| | Amgen | | non-hodgkins lymphoma |
| Hu M195Mab | Protein Design Labs | Anti-CD33 | Cancers |
| MDX-210 | Immuno-Designed Molecules | ND | Cancers |
| BEC2 | Imclone | anti-GD3 | Cancers (glioblastoma, |
| Mitumomab | | | malignant melanoma, |
| | | | neuroblastoma) |
| Oregovomab | Altarex | anti-CA125 | Ovarian cancer |
| OVAREX | | | |
| Ecromeximab | Kyowa-Hakko | anti-GD3 | Malignant melanoma |
| KW-2971 | | | |
| ABX-EGF | Abgenix | EGF | Cancers |
| MDX010 | Medarex | Anti-CD4R | Cancers |
| XTL 002 | XTL | ND | Anti-viral: HCV |
| | biopharmaceuticals | | |
| H11 SCFV | viventia | ND | Cancers |
| | biotech | | |
| 4B5 | viventia | anti-GD2 | Cancers |
| | biotech | | |
| XTL 001 | XTL | ND | Anti-viral: HBV |
| | biopharmaceuticals | | |
| MDX-070 | MEDAREX | Anti-PSMA | Prostate cancer |
| TNX-901 | TANOX | anti-IgE | Allergies |
| IDEC-114 | IDEC | Protein C | Non-hodgkins lymphoma |
| | | inhibition | |

For the purposes of the invention, by “antibody selective for activating antibody Fc region receptors (FcRs)” or “antibody the Fc region of which selectively binds activating antibody Fc region receptors (FcRs)” is meant any antibody which possesses the ability to recruit activating FcRs but which possesses an ability to engage inhibitory FcRs which is reduced or even null, relative to the same antibody possessing a native Fc region.

Thus, the antibodies of the invention possess the ability to recruit, i.e. to bind by their Fc region, the activating FcR receptors, but their ability to engage, i.e. to bind by their Fc region, the inhibitory FcR receptors, is reduced or eliminated relative to the same antibody possessing a native Fc region.

By activating FcR is meant FcγRIIIA, FcγRTIIB, FcyRIIA, FcγRIA and FcγRIB, FcγR, FcγRI and the human equivalent of FcγRIV described in mice. By inhibitory FcR, is meant the FcγRIIBs.

During Step c), it is possible to select the antibodies, i.e. different antibody compositions, for which the binding to the FcRs is reduced relative to the same antibody possessing a native Fc region. This reduction can for example be measured by quantifying the number or percentage of cells expressing the FcRs to which the antibodies of the invention are bound, and by comparing this number or this percentage to that of cells expressing the FcRs to which the antibodies possessing a native Fc region are bound.

In a preferred embodiment of the invention, in Step c) the antibodies, i.e. different antibody compositions, for which the binding to the inhibitory FcγRs is eliminated relative to the same antibody possessing a native Fc region, are selected according to a method similar to that described previously. Thus, the antibodies prepared according to the method of the invention possess an Fc region which binds to activating FcRs but does not bind to inhibitory receptors, whereas the same antibody produced by the same biological system but possessing an Fc region not modified according to the method of the invention binds, by its Fc region, to activating FcγRs and inhibitory FcγRs.

In a preferred embodiment of the invention, the His 310 and His 435 residues are replaced with a lysine residue.

In a preferred embodiment of the invention, the His 310 and His 435 residues are replaced by site-directed mutagenesis or by molecular evolution.

In another embodiment, the His 310 and His 435 residues are modified by means of a chemical treatment, for example by treatment with DEPC (diethylpyrocarbonate, which is a histidine modifying agent).

The Applicant surprisingly found that the mutation of the two particular histidine residues His 310 and His 435 situated at the CH2/CH3 interface of an antibody by a residue chosen from lysine, alanine, glycine, valine, leucine, isoleucine, proline, methionine, tryptophan, phenylalanine, serine or threonine, and preferably by lysine, has a major impact on the binding of the antibody thus modified to inhibitory FcRs, and in particular to the FcγRIIBs, and has a moderate effect on its binding to activating FcRs, and in particular to the FcγRIIIAs. The Applicant has demonstrated that the antibody of the invention thus modified no longer, or virtually no longer, binds the human inhibitory FcγRIIB receptor in an in vitro binding test whereas its binding to the activating FcγRIIIA and FcγRIIA receptors is only very partially inhibited relative to the non-mutated antibody (see below). This mutated antibody is an antibody capable of engaging the activating receptors (FcγRIIA and FcγRIIIA) involved in the ADCC-type cytotoxicity by avoiding engaging the inhibitory FcyRIIB. Such an antibody therefore makes it possible to induce an ADCC against target cells (red blood cells, tumour cells, allo-reactive cells, cells infected with microbial pathogens) without this ADCC being negatively modulated following the engagement of the FcyRIIB. Such an antibody also makes it possible to optimize the antigen presentation by dendritic cells due to the fact that the immune complexes containing this mutated antibody are captured only by the activating FcγRs expressed on the dendritic cells, without activating the inhibitory FcγRIIBs, also present on these cells.

This “differential” behaviour on the activating and inhibitory receptors is an essential asset for using the antibodies of the invention therapeutically, in particular within the framework of cancerous or infectious pathologies. In fact, such an antibody is capable of inducing ADCC mechanisms via the activating FcγRs without the latter being negatively modulated by the inhibitory FcγRs. Moreover, the Applicant has found that such an antibody only recruits the activating FcγRs on the dendritic cells and does not recruit the inhibitory FcγRs at the surface of the same cells. It has recently been shown that the engagement of the inhibitory FcγRs at the surface of the dendritic cells has a negative effect on the maturation of these cells and on their ability to effectively present an antigen to the effector T lymphocytes, rendering these dendritic cells tolerogenic. If such an antibody complexed to an antigen recognizes only the activating FcγRs expressed at the surface of the dendritic cells and not the inhibitory FcγRs, the antigen presentation by the dendritic cells which results from this is optimal and the effect of activation of the optimized specific immune response is optimized.

Advantageously, the Fc region of the antibody originating from the method of the invention binds to activating Fc receptors whereas it does not bind to inhibitory Fc receptors.

Advantageously, the antibody of the method of the invention does not recruit inhibitory Fc receptors, in particular the inhibitory Fc receptors expressed by the B lymphocytes.

Particularly advantageously, the antibody of the method of the invention does not recruit inhibitory Fc receptors but binds a molecule at the surface of the B lymphocytes.

Advantageously, the antibody of the invention does not recruit inhibitory Fc receptors but binds a molecule at the surface of tumour cells, in particular at the surface of tumorous B lymphocytes.

Advantageously, the antibody of the invention does not recruit inhibitory Fc receptors but binds a molecule at the surface of B-CLL tumorous lymphocytes. Advantageously, the antibody of the invention does not recruit inhibitory Fc receptors but binds the CD20 molecule at the surface of B-CLL tumorous lymphocytes.

Particularly advantageously, the antibody of the method of the invention binds to the FcyRIII receptor (A and B isoforms) and/or to the FcγRIIA receptor and/or to the FcγRI receptor (A and B isoforms), whereas it does not bind to the FcγRIIB receptors (B1 and B2 isoforms). In a particular embodiment, the FcγRIIB receptor is the FcyRIIB1 receptor.

Preferably, the receptors involved in the method of the invention are human receptors.

In a particular embodiment of the invention, the receptors of the Fc region of the antibodies are located on the monocytes, macrophages, dendritic cells, NK cells, B lymphocytes, monocytes, macrophages and B lymphocytes.

In another particular embodiment of the invention, the inhibitory receptors of the Fc region of the antibodies are located on tumour cells, such as malignant melanoma cells, tumorous B lymphocytes such as lymphomas, B-CLL (B chronic lymphoid leukaemia) cells and myeloma cells.

Advantageously, the antibody of the method of the invention binds, by its variable region, the CD20 molecule at the surface of B-CLL tumorous lymphocytes. Such an antibody of the invention does not recruit inhibitory Fc receptors, in particular at the surface of the B-CLL tumorous lymphocytes.

Advantageously, the antibodies utilized in the m of the invention are monoclonal antibodies. These monoclonal antibodies can be produced by any appropriate biological system, in the form of monoclonal antibody compositions which can contain at least one excipient or pharmaceutically acceptable vehicle such that these compositions can be formulated in order to allow pharmaceutical administration, for a prophylactic and/or therapeutic purpose. By “biological system” is meant animal or plant cell lines transfected using one or more vectors in order to express said antibody, plants or non-human transgenic animals, as well as any hybridoma, heterohybridoma. Among the cells, it is possible to choose cells originating from cell lines, transfected using a vector comprising the gene coding for said antibody, for example eukaryotic or prokaryotic cells, in particular cells of mammals, insects, plants, bacteria or yeast. Preferentially, rat myeloma cells such as YB2/0 (ATCC No. CRL 1662) are used.

It is also possible to use CHO cells, in particular CHO-K, CHO-Lec1O, CHO-Lec1O CHO Pro-5, CHO dhfr- or other cell lines from Wil-2, Jurkat, Vero, Molt-4, COS-7, 293-HEK, K6H6, NSO, SP2/0-Ag 14 and P3X63Ag8.653, PERC6 or BHK.

Another object of the invention is the use of an antibody for which each of the His 310 and His 435 residues (Kabat numbering) of its Fc region has been replaced by a residue chosen from lysine, alanine, glycine, valine, leucine, isoleucine, proline, methionine, tryptophan, phenylalanine, serine or threonine, or an antibody produced by the method of the invention, for obtaining a medicament intended for anti-infection and anti-tumour vaccination, the antibody of the invention being engaged in an immune complex which can be bound only by the activating FcRγs of the antigen-presenting cells (monocytes, macrophages, dendritic cells, epidermal Langerhans' cells, B lymphocytes).

Preferably, for the preparation of such a medicament an antibody for which each of the His 310 and His 435 residues (Kabat numbering) of its Fc region has been replaced with a lysine residue is used.

Another subject of the invention relates to the use of an antibody, each of the His 310 and His 435 residues (Kabat numbering) of which is replaced by a residue chosen from lysine, alanine, glycine, valine, leucine, isoleucine, proline, methionine, tryptophan, phenylalanine, serine or threonine, or an antibody produced by the method of the invention, for obtaining a medicament intended for the treatment of cancers, and more particularly for the treatment of lymphomas, leukaemias, myelomas, sarcomas, solid tumours such as mammary carcinomas, colorectal tumours, pancreatic tumours, prostate tumours, stomach tumours, pulmonary tumours, ovarian tumours, cervical tumours, ocular tumours, thyroid tumours, tumours of the ENT sphere, malignant melanoma, nerve tumours such as glioblastomas, and neuroblastomas.

Preferably, for the preparation of such a medicament, an antibody for which each of the His 310 and His 435 residues (Kabat numbering) of its Fc region has been replaced with a lysine residue is used.

Another subject of the invention is the use of an antibody, each of the His 310 and His 435 residues (Kabat numbering) of which is replaced by a residue chosen from lysine, alanine, glycine, valine, leucine, isoleucine, proline, methionine, tryptophan, phenylalanine, serine or threonine, or an antibody produced by the method of the invention, for obtaining a medicament intended for the treatment of infectious diseases such as infection with HIV, HBV, HCV, RSV (respiratory syncytial virus), SARS virus, rotavirus, influenza virus, variola, bacterial infections such as infections caused by Koch's bacillus, meningococci, Criptoccocus neoformans, Clostridium, the bacteria responsible for botulism, anthrax, tetanus, tuberculosis, and enterococcal infections.

Preferably, for the preparation of such a medicament an antibody, for which each of the His 310 and His 435 residues (Kabat numbering) of its Fc region has been replaced with a lysine residue, is used.

Finally, a last aspect of the invention relates to an antibody composition for which the binding to inhibitory FcRs is reduced by at least 30%, preferably at least 50%, 70%, 80% or also at least 90% or 100% relative to the same antibody possessing a native Fc region.

Preferably, the composition of the invention is a composition the Fc region of the antibodies of which possesses an ability to bind to the inhibitory receptors which have been eliminated relative to the same antibody possessing a native Fc region.

Particularly advantageously, the composition of the invention is an anti-CD20 antibody composition.

Other aspects and advantages of the invention are described in the examples which follow, which must be considered as illustrative and do not limit the scope of the invention.

### DESCRIPTION OF THE FIGURES

FIG. 1: Effect of the modification by diethylpyrocarbonate of the histidines of the anti-RhD mAb T125(YB2/0) on the ability of the antibody to recruit human FcγRs.

FIG. 2: Effect of the mutation of the histidines 310 and 435 of the anti-RhD mAb T125(YB2/0) to lysines on the ability of the antibodies to bind human FcγRs.

FIG. 3: Effect of the mutation of the histidines 310 and 435 of the anti-RhD mAb T125(YB2/0) on the ability of the mAb to induce a production of IL-2 dependent on the human FcγRIIIAs.

FIG. 4: Comparison of the binding of T125(YB2/0), the double mutant T125(YB2/0) His³¹⁰Lys/His⁴³⁵Lys and T125 (CHO) to human FcγRIIIAs and FcγRIIBs.

FIG. 5: Effect of the mutation of the histidines 310 and 435 of the anti-RhD monoclonal antibody T125(YB2/0) on the ability of the monoclonal antibody to induce an ADCC dependent on the human FcγRIIIAs compared to the native (wild-type) antibody produced in YB2/0 and the native (wild-type) antibody produced in CHO.

FIG. 6: Representation at the surface of the crystallographic structures of the Fc fragments of T125(YB2/0) in the presence of Zinc (A) and the double mutant T125H310K-H435K(YB2/0) (B).

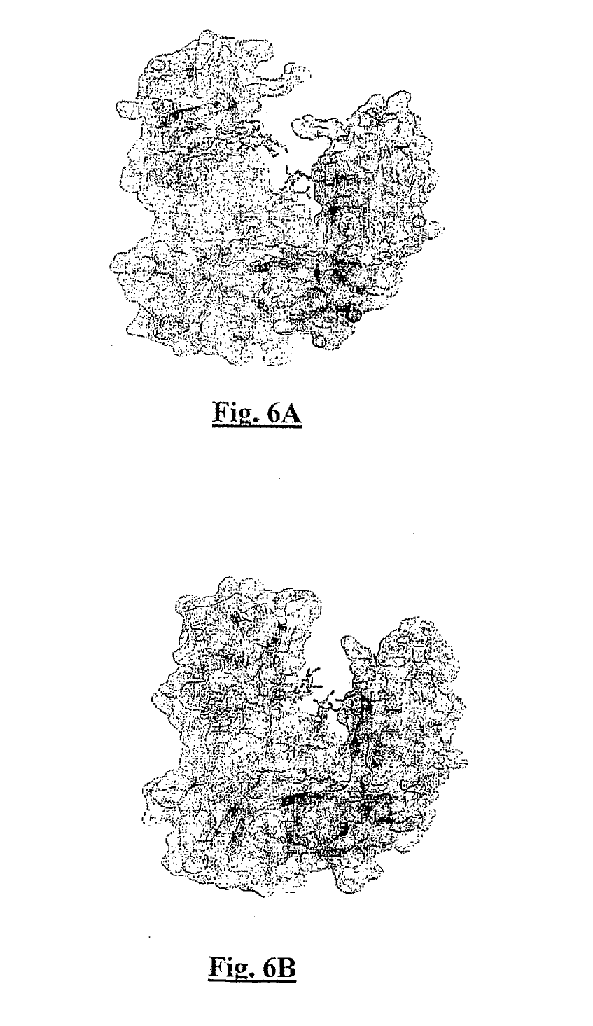

### EXAMPLES

### Example 1

### Obtaining an Antibody Carrying the Double Mutation His310-435Lys

The line YB2/0 (rat myeloma, line ATCC No. CRL 1662) transfected and producing the EMAB5 antibody (described in the document WO 2005/040216), which is a human IgG1 (γ) directed against the Rh(D) antigen, was adapted for culture in medium without serum.

EMAB5 was purified by affinity chromatography on Sepharose-protein A.

By HPCE-LIF, it was shown that the majority glycanic structure is a biantenna-type oligosaccharide, containing approximately 25% fucose.

### Preparation of the Fc Fragment:

The purified EMAB5 antibody is dialyzed overnight against 50 mM Tris buffer, pH 8.0. The antibody solution, adjusted to 50 mM CaC12 and 10 mM cysteine, is incubated for 30 minutes at 37° C. before adding the trypsine solution (1 mg/ml) in an enzyme/substrate ratio of 1/25.

After incubation for 5 hours at 37° C., the reaction is stopped by the addition of diisopropyl fluorophosphate (1 mM final). The hydrolysate is dialyzed overnight against 50 mM Imidazole buffer, pH 7.8.

For the purification of the Fc fragment, the dialyzed hydrolysate is brought into contact with protein L-affarose at a rate of 1 ml of gel per 3.6 mg of antibodies. After incubation for 4 hours at ambient temperature under stirring, the gel is loaded in a column and washed with 50 mM Imidazole buffer, pH 7.8. The effluent and the washing buffer which contain the Fc fragments are combined and concentrated by centrifugation on a Vivaspin 20 using the conditions described by the manufacturer.

### Site-Directed Mutagenesis:

The expression vector containing the cDNA encoding the amino acid sequence of the heavy chain of the anti-Rh(D) antibody EMAB5, served as a matrix for carrying out a double site-directed mutagenesis by PCR (“PCR-based site-directed mutagenesis”). The following four nucleotide substitutions were introduced:
C1229A and C1301G for changing the His338 residue to Lys (position 310 according to Kabat numbering), i.e. CAC→AAG;
C1674A and C1676G for the mutation of the His463 residue to Lys (position 435 according to Kabat numbering), i.e. CAC→AAG.

The heavy chain of the mutated antibody has, for a nucleotide sequence, the sequence SEQ ID NO: 1 and, for a peptide sequence SEQ ID NO: 2 (the mutated amino acids appear in the sequence SEQ ID NO: 2 at position 338 and 463 respectively for the amino acids Lys310 and Lys435). The numbering takes account of the leader sequences (338 and 463) or it does not (in the latter case, it is the so-called Kabat numbering which was used: 310 and 435).

The YB2/0 cells, co-transfected by electroporation with the mutated vector EMAB5-H-K338-K463-1 and the vector EMAB5-dhfr-K-SpeI coding for the light chain of the EMAB5 antibody, are cultured in RPMI medium supplemented with 5% dialyzed FCS, 0.5% G418 and 25 nM of Methotrexate (MTX). The clones secreting the highest level of human IgGs are cultured on 24-well plates in medium without MTX. The supernatants, collected after culture for 7 days, are used to carry out the tests described below.

### Example 2

### Effect of the Modification of the Histidines at the CH2/CH3 Interface of an Anti-RhD Monoclonal IgG1 by Diethylpyrocarbonate (DEPC) on Human IgG1/FcRγ Interactions

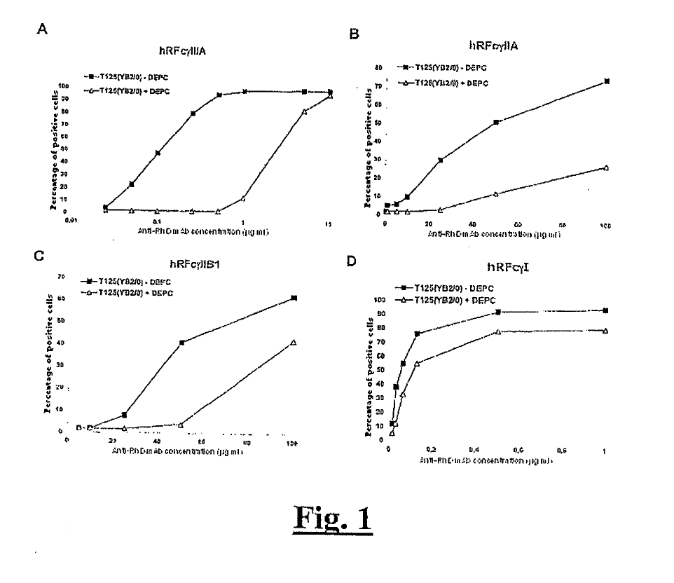

In order to study the impact of a modification of the histidines of a human IgG1 on the human IgG1/FcγR interactions, the anti-RhD monoclonal antibody, T125(YB2/0) was treated with diethylpyrocarbonate (DEPC). The DEPC modifies the histidine residues by the creation of a covalent bond between a nitrogen atom of the histidine ring and a carbon atom of the DEPC molecule. The monoclonal antibodies, treated or not treated with DEPC, are fractionated on a column of protein A-Sepharose. Histidine 435 being important for binding IgG to the protein A, the fraction of monoclonal antibodies treated with DEPC and not retained on protein A corresponds to IgG1s at least the His⁴³⁵ residues of which have been modified. The monoclonal antibodies T125(YB2/0) not treated or treated with DEPC and not retained on protein A were compared for their binding to different types of human FcRγs (FIG. 1). The binding of the anti-RhD monoclonal antibody T125(YB2/0), treated or not treated with diethylpyrocarbonate (DEPC) to human FcγRIIIA (A), FcγRIIA (B), FcγRTIB1 (C) and FcγRI (D) (hFcγR) was analyzed by indirect immunofluorescence. The indicator cells Jurkat-huFcγRIIIA (A), K562 (B), IIA. 1.6-huFcγRIIB1 (C) and Tf2-13 (D) are incubated with different concentrations of T125(YB2/0) treated or not treated with DEPC. The binding of the monoclonal antibody is detected by mouse anti-human IgG (H+L) F(ab′)₂ coupled to FITC). T125(YB2/0) binds to the FcγRIIIAs at a low concentration (from 0.05 μg/ml) and exhibits very significant binding at 0.5 μg/ml (95% positive cells). When this monoclonal antibody is treated with DEPC, its binding is reduced at a high concentration (between 1 and 5 μg/ml) (approximately 90% reduction at 1 μg/ml) and becomes marginal at a low concentration (between 0.025 μg/ml and 0.05 μg/ml) (FIG. 1A). The treatment with DEPC also induces a 76% and 64% reduction in the binding of T125 (YB2/0) to the FcγRIIAs (at 50 μg/ml and 100 μg/ml, respectively) (FIG. 1B). Similarly, the binding of T125(YB2/0) treated with DEPC to the FcγRIIB1 is reduced: at 50 μg/ml, the binding of T125(YB2/0) treated with DEPC is marginal whereas approximately 40% of cells are positive for the same concentration of untreated T125(YB2/0) (FIG. 1C). On the other hand, the modification of the mAb by the DEPC has a moderate effect on its binding to the FcγRIs. The treatment with DEPC induces a 15% reduction in the binding of T125(YB2/0) to the FcγRI at 0.5 μg/ml and 1 μg/ml. (FIG. 1D).

In conclusion, the blocking of the histidines present in T125(YB2/0) by the DEPC reduces its binding to the FcγRIIIAs (FIG. 1A), FcγRIIAs (FIG. 1B), FcγRIIBs (FIG. 1C) and, to a lesser extent, to the human FcγR1 (FIG. 1D).

### Example 3

### Effect of Mutations of the His435 and His³¹⁰ Residues of an Anti-RhD Monoclonal IgG1 on Human IgG1/FcγR Interactions

The preceding results indicate that the modification of the His residues of a monoclonal IgG1 affect its interactions with the human FcγRs. However, the treatment with DEPC does not make it possible to determine which His's have been modified.

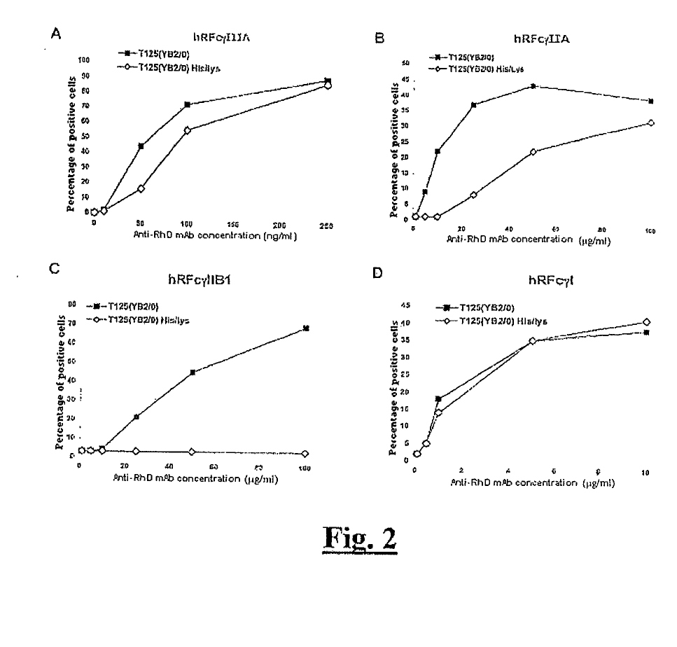

Structural studies have shown the importance of the His⁴³⁵ and His³¹⁰ residues situated on either side of the hinge region of the IgG1s in the IgG1/FcγR interactions. We therefore studied the effect of the mutation of the His⁴³⁵ and His³¹⁰ residues of T125(YB2/0) to lysine on the binding of the monoclonal antibody to the human FcRγs (FIG. 2). The binding of the monoclonal antibody T125(YB2/0) or of the double mutant T125(YB2/0) His³¹⁰Lys/His⁴³⁵Lys to the human FcγRIIIAs (A), FcγRIIAs (B), FcγRIIB1s (C) and FcγRIs (D) (hFcγR) was analyzed by indirect immunofluorescence. The indicator cells Jurkat-huFcγRIIIA (A), K562 (B), IIA.1.6-huFcγRTIB1 (C) and Tf2-13 (D) are incubated with different concentrations of T125(YB2/0) or of the double mutant T125(YB2/0) His³¹⁰Lys/His⁴³⁵Lys. The binding of the mAbs is detected by mouse anti-human IgG (H+L) F(ab′)₂ fragments coupled to FITC). The binding of the double mutant T125(YB2O) His³¹⁰Lys/His⁴³⁵Lys to the human FcγRIIIAs expressed by the Jurkat-CD16 cells relative to that of the non-mutated monoclonal antibody is slightly reduced (FIG. 2A). This binding is also reduced when the experiments are carried out with indicator cells (K562) expressing the human FcγRIIA (FIG. 2B). By contrast, the mutation of the His³¹⁰ and His⁴³⁵ residues of T125(YB2/0) completely eliminates the binding of this antibody to the human FcγRIIB1 (FIG. 2C). The binding of T125(YB2O) His310Lys/His435Lys to the human FcγRIs is not affected (FIG. 2D).

### Example 4

### Effect of Mutations of the His435 and His³¹⁰ Residues of an Anti-RhD Monoclonal IgG1 on its Effector Properties

We analyzed the effect of the mutation of the His³¹⁰ and His⁴³⁵ residues of T125(YB2/0) on one of the activating effector functions of this antibody.

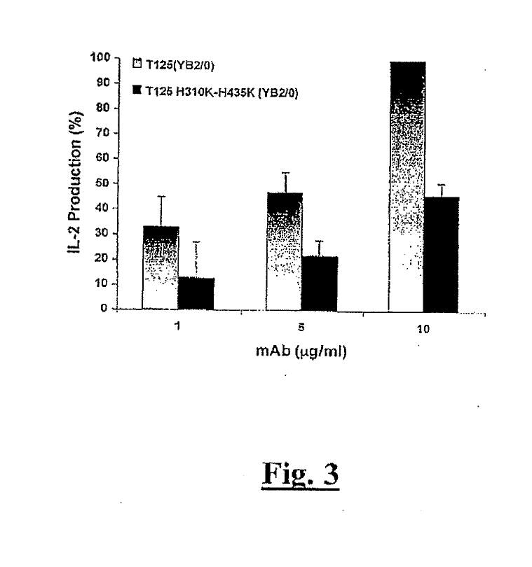

The abilities of the double mutant and of the unmutated monoclonal antibody to induce a production of IL-2 by the Jurkat-huFcγRIIIA FcγRIIIA⁺ cells were compared by ELISA (FIG. 3). In order to standardize the results originating from three different experiments, the production of IL-2 induced by different doses of monoclonal antibody is reported as a percentage of that induced by 10 μg/ml of T125(YB2/0). This dose of T125 (YB2/0) corresponds to the maximum production of IL-2 detected in all the experiments carried out. The production of IL-2 induced by 1, 5 and 10 μg/ml of mutated T125(YB2/0) is reduced by 61%, 53%, and 54% respectively, relative to the release of IL-2 induced by the same doses of the unmutated monoclonal antibody (FIG. 3). The Jurkat-huFcγRIIIA (FcγRIIIA⁺) cells are stimulated for 15 hours at 37° C. by different concentrations of T125(YB2/0) or of the double mutant T125(YB20) His³¹⁰Lys/His⁴³⁵Lys, in the presence of rabbit anti-human IgG(H+L) F(ab′)₂ fragments allowing the aggregation of the anti-RhD human monoclonal antibodies. The production of IL-2 by the Jurkat-huFcγRIIIA cells is then detected by ELISA. The production of IL-2 induced in the presence of different concentrations of the two mAbs is reported as a percentage of that induced by 10 μg/ml of T125(YB2/0)).

These results indicate that the modification of the His³¹⁰ and His⁴³⁵ residues at the CH2/CH3 interface of a monoclonal IgG1 partially reduces the binding of this mAb to the human FcγRIIIAs as well as its ability to induce a production of cytokine dependent on these activating FcRγs. Nevertheless, the double mutant monoclonal antibody T125(YB20) His³¹⁰Lys/His⁴³⁵Lys is also capable of recruiting activating FcγRIIIAs and of inducing activating effector functions dependent on these receptors.

### Example 5

### Comparison of the Engagement Profiles of the Human FcγRs of T125 (YB2/0), the Double Mutant T125(YB2/0) His³¹⁰Lys/His⁴³⁵Lys and T125 (CHO)

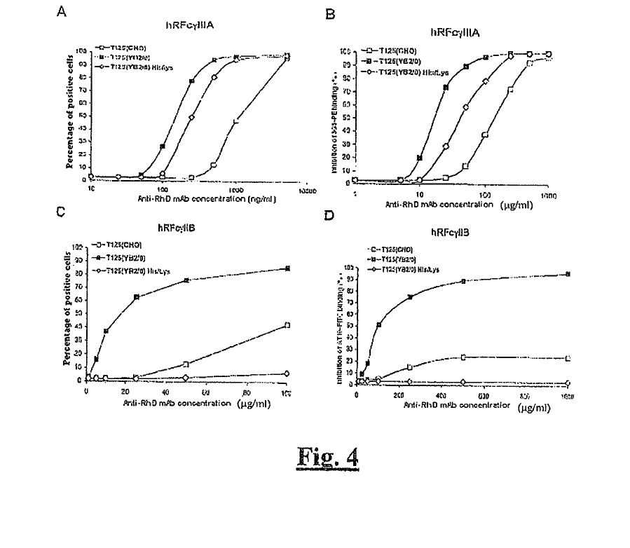

The preceding experiments show that the double mutant monoclonal antibody T125(YB2O) His³¹⁰Lys/His⁴³⁵Lys is an antibody capable of engaging the activating receptors (FcγRIIA and FcγRIIIA), but the ability of which to bind the inhibitory FcγRIIBs is eliminated. In order to better characterize the behaviour of this monoclonal antibody, we compared its binding to human FcγRIIIAs and FcγRIIBs with that of T125 (CHO) for which the structural properties and the profile of engagement with the different types of FcγR have been precisely defined (FIG. 4). (A) and (C), the binding of the mAbs T125(YB2/0), T125 (YB2/0) His³¹⁰Lys/His⁴³⁵Lys and T125 (CHO) to the human FcγRIIIAs (A), and FcγRIIB1s (C) was analyzed by indirect immunofluorescence. The indicator cells Jurkat-huFcγRIIIA (A), or IIA.1.6-huFcγRTIB1 (C) are incubated with different concentrations of T125(YB2/0), T125(YB2/0) His³¹⁰Lys/His⁴³⁵Lys and T125 (CHO). The binding of the monoclonal antibodies is detected by mouse anti-human IgG (H+L) F(ab′)₂ fragments coupled to FITC.

(B) and (D), the abilities of the monoclonal antibodies T125(YB2/0), T125(YB2/0) His³¹⁰Lys/His⁴³⁵Lys and T125 (CHO) to inhibit the binding of the 3G8-PE antibodies (anti-human FcγRIIIA/IIIB) (B) or AT10-FITC (anti-human FcγRIIA/IIB) (D) were compared. The indicator cells Jurkat-huFcγRIIIA (B) or IIA. 1.6-huFcγRIIB1 (D) are incubated with different concentrations of T125(YB2/0), or T125(YB2/0) His³¹⁰Lys/His⁴³⁵Lys or T125 (CHO), then with 40 ng/ml of 3G8-PE (B) or 40 ng/ml of AT10-FITC (D), respectively. The percentage of inhibition of the binding of 3G8-PE or AT10-FITC as a function of the concentration of the competing monoclonal antibodies is calculated: T125 (CHO), which is more fucosylated than T125(YB2/0), is capable of inducing inhibiting functions dependent on the FcγRIIB1s. On the other hand, it only weakly binds the human FcγRIIIAs and is a poor inductor of the activating functions dependent on the FcγRIIIAs.

Indirect immunofluorescence experiments show that, although the binding of the double mutant monoclonal antibody T125(YB20) His³¹⁰Lys/His⁴³⁵Lys to the FcRγIIIAs are slightly reduced relative to that of T125(YB2/0), its binding remains much greater than that of T125 (CHO) (FIG. 4A). We confirmed this difference in binding to the human FcγRIIIAs between the double mutant of T125(YB2/0) and T125 (CHO) by competition experiments using 40 ng/ml of 3G8-PE, which blocks the human FcγRIIIA and FcγRIIIB binding sites (FIG. 4B). Concentrations of approximately 15 μg/ml in the case of T125(YB2/0) and of 40 μg/ml in the case of T125(YB2/0) His³¹⁰Lys/His⁴³⁵Lys are required in order to induce a 50% inhibition of the binding of the monoclonal antibody 3G8-PE to the FcRγIIIAs expressed at the surface of the Jurkat-huFcγRIIIA cells. By contrast, a dose of T125 (CHO) greater than 100 μg/ml (approximately 130 μg/ml) is necessary to achieve a 50% inhibition of the binding of 3G8-PE.

The indirect immunofluorescence experiments show that the binding of the double mutant T125(YB20) His³¹⁰Lys/His⁴³⁵Lys to the HA.1.6-huFcγRIIB1 (FcγRIIB1⁺) indicator cells is completely eliminated, whereas that of T125 (CHO) is maintained despite a reduction in the ability of this last mAb to bind to the FcRγIIB1s relative to T125(YB2/0) (FIG. 4C). Similarly, whatever the concentration tested, the double mutant T125(YB20) His³¹⁰Lys/His⁴³⁵Lys is incapable of inhibiting the binding of 40 ng/ml of the AT10-FITC antibody (anti-human FcγRIIA/FcγRIIB mAb) to the HA.1.6-huFcRγIIB1 cells, whereas T125 (CHO) is capable of inducing an inhibition of the binding of the AT10-FITC antibody to the FcγRIIB1s, although the latter is less than that induced by T125(YB2/0) (FIG. 4D).

Therefore, these immunofluorescence experiments show that the double mutant T125(YB2O) His³¹⁰Lys/His⁴³⁵Lys has a behaviour different from that of T125 (CHO), in terms of interactions with the FcγRs: T125(YB20) His³¹⁰Lys/His⁴³⁵Lys effectively recruits the activating FcγRIIIAs. On the other hand, this monoclonal antibody is incapable of engaging the FcγRIIBs.

### Example 6

### Comparison of the ADCC Obtained with the T125 Antibody (YB2/0), the Double Mutant T125 Antibody (YB2/0) His³¹⁰Lys/His⁴³⁵Lys and the T125 Antibody (CHO)

The ability to induce an ADCC lysis of the Rhesus positive erythrocytes induced by different anti-Ds in the presence of mononucleated cells (source of effector cells) and of Tegeline (2500 μg/ml) was compared for different anti-D antibodies. The AD1 antibody is an anti-D antibody triggering no ADCC response and therefore serves as a negative control. The T125 antibody was expressed in two different cell types: YB2/0 (producing the T125 antibody (YB2/0) and CHO (producing the T125 antibody (CHO)). Moreover, the T125 antibody was mutated to in order to replace each of its His 310 and His 435 residues with lysine residues (T125(YB2/0) His³¹⁰Lys/His⁴³⁵Lys).

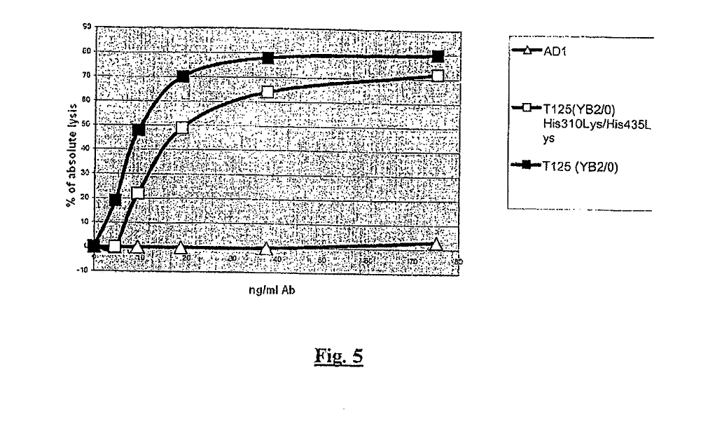

The results appear in FIG. 5: the mutated histidine antibody (H310K H435K) induces an ADCC of the Rhesus positive erythrocytes slightly less than that obtained with the non-mutated T125 antibody (YB2/0), but much greater than AD1. It should be noted that under these experimental conditions, the anti-D control antibody expressed in CHO induces very little if any ADCC (results not shown).

### Example 7

### Structural Impact of the Mutation of the Histidines 310 and 435 to Lysine

Comparison of the crystallographic structures of the Fc fragments of T125(YB2/0) and T125H310K-H435K(YB2/0) (cf Example 6) shows that the mutations of histidines 310 and 435 to lysines have not modified the general conformation of the Fc fragment. In the document WO 2005/040216 (which is incorporated here by way of reference), the Applicant shows that the non-mutated antibodies, therefore carrying the His 310 and His 435 residues, possess the ability to bind Zinc cations (Zn²⁺). The Fc fragment carrying the lysines has lost its ability to bind the Zn²⁺ cations; however, the position of the side chain of these residues as well as their dimensions are superimposed with those of the histidine residues of the Fc fragment of T125 (YB2/0). This fact is important for use of this type of antibody in human therapeutics since this suggests that the binding of the T125 antibody H310K-H435K(YB2/0) to the FcnR, and therefore its catabolism, will not be modified. By contrast, the mutation of the histidine 310 and 435 residues to lysines has an impact on the orientation of the CH2 domains of the Fc fragment. In fact, comparison of the crystallographic structures of the Fc fragments of T125(YB2/0) (in the presence of zinc) and the double mutant T125H310K-H435K(YB2/0) show that the latter has a more closed conformation in which the two CH2 domains are closer to each other relative to the CH2 domains of the Fc fragment of T125 (YB2/0). This difference in conformation could be at the origin of the impact of the H310K and H435K mutations on the binding of the antibody to the human FcγRs (cf Examples 3, 4 and 5).

1. Method for preparing an antibody possessing the ability to recruit activating FcRs, but the ability of which to recruit inhibitory FcRs is reduced relative to the same antibody possessing a native Fc region, comprising the following steps:
a) obtaining monoclonal antibodies from a hybridoma, heterohybridoma, or any animal, plant or human cell line,
b) replacing each of the His 310 and His 435 residues (Kabat numbering) of the Fc region of said antibody with a residue chosen from lysine, alanine, glycine, valine, leucine, isoleucine, proline, methionine, tryptophan, phenylalanine, serine or threonine,
c) selecting the antibodies for which the binding to inhibitory FcRs is reduced by at least 30%, preferably at least 50%, 70%, 80% or also at least 90% relative to the same antibody possessing a native Fc region.

2. Method according to claim 1, characterized in that in Step c), the antibodies for which the binding to inhibitory FcRs is eliminated are selected.

3. Method according to claim 1, characterized in that said chosen residue is lysine.

4. Method according to claim 1, characterized in that the antibody originating from Step b) is expressed in YB2/0 (ATCC No. CRL 1662).

5. Method according to claim 1, characterized in that said Fc region of said antibody selected in Step c) binds to activating FC receptors whereas it does not bind to inhibitory Fc receptors.

6. Method according to claim 1, characterized in that said antibody binds to the FcγRIII receptor (A and B isoforms) and/or to the FcγRIIA receptor and/or to the FcγRI receptor (A and B isoforms), whereas it does not bind to the FcγRIIB receptor (B1 and B2 isoforms).

7. Method according to claim 1, characterized in that said receptors are human receptors.

8. Method according to claim 1, characterized in that said receptors of the Fc region of the antibodies are located on the dendritic cells, the monocytes, the macrophages, the B lymphocytes.

9. Method according to claim 1, characterized in that said inhibitory receptors of the Fc region of the antibodies are located on tumour cells, such as malignant melanoma cells, tumorous B lymphocytes such as lymphomas and B-CLLs and myeloma cells.

10. Method according to claim 1, characterized in that said antibody binds the CD20 molecule at the surface of B-CLL tumorous lymphocytes.

11. Method according to claim 1, characterized in that said residues are replaced by site-directed mutagenesis or by molecular evolution.

12. Use of an antibody produced by means of the method as defined in claim 1, for obtaining a medicament intended for anti-infection and anti-tumour vaccination, said antibody being engaged in an immune complex which can only be bound by the activating FcγRs of the antigen-presenting cells (monocytes, macrophages, dendritic cells, epidermal Langerhans cells, B lymphocytes).

13. Use of an antibody produced by means of the method as defined in claim 1, for obtaining a medicament intended for the treatment of cancer.

14. Use according to claim 13, for obtaining a medicament intended for the treatment of a cancer chosen from the lymphomas, leukaemias, myelomas, sarcomas, solid tumours such as mammary carcinomas, colorectal tumours, pancreatic, prostate tumours, stomach tumours, pulmonary tumours, ovarian tumours, cervical tumours, ocular tumours, thyroid tumours, tumours of the ENT sphere, malignant melanomas, nerve tumours such as glioblastomas, neuroblastomas.

15. Use of an antibody produced by means of the method as defined in claim 1, for obtaining a medicament intended for the treatment of infectious diseases such as infection by HIV (human immunodeficiency virus), HBV (hepatitis B virus), HCV (hepatitis C virus), RSV (respiratory syncytial virus), SARS virus (atypical pneumonia virus), rotavirus, influenza virus, variola, bacterial infections such as the infections caused by Koch's bacillus, meningococci, Criptoccocus neoformans, Clostridium, tuberculosis, and enterococcal infections.
